(19) United States
(12) Patent Application Publication
(10) Pub. No.: US 2021/0239622 A1
(43) Pub. Date: Aug. 5, 2021

(54) Calixarene Indicator

(71) Applicant: Paul SPIEL, Dresden (DE)

(72) Inventor: Paul D Spiel, Lindon, UT (US)

(21) Appl. No.: 17/054,299

(22) PCT Filed: Jun. 3, 2019
(86) PCT No.: PCT/US19/35188

§ 371 Date: Nov. 10, 2020

Related U.S. Application Data
(60) Provisional application No. 62/681,542, filed on Jun. 6, 2018.

(51) Int. Cl. G01N 21/80; G01N 31/22; C07C 39/17

(52) U.S. Cl. CPC G01N 21/80; G01N 31/221; G01N 2021/7759; C07C 2603/90; C07C 39/17

(57) ABSTRACT

A pyrogallarene solution and a method for using a calixarene as an indicator are disclosed. The calixarene may be a pyrogallarene, and the pyrogallarene may be a pyrogallol[4]arene. The pyroallol[4]arene may be C-alkyl pyrogallol[4]arene. The calixarene indicator having a dynamic chromophore may be used to assess pH, metal content, or water content.

### CROSS-REFERENCE TO RELATED APPLICATIONS

This application claims priority to U.S. provisional patent application 62/681,542 filed 6 Jun. 2018.

### BACKGROUND OF THE INVENTION

### 1. The Field of the Invention

The invention relates to indicators. In accordance with various embodiments it relates to the visual assessment of the pH in various solutions.

### 2. The Relevant Technology

Indicators are commonly used to probe solutions. Often the compounds used for indication have multiple double bonds and extended conjugation, resulting in delocalization of pi electrons. As pi electrons become more delocalized they absorb at higher wavelengths. All wavelengths of the electromagnetic spectrum can be probed with sufficient instrumentation, but if a compound absorbs or emits light in the visible spectrum it can be perceived visually. Visual perception occurs in the range of 380 to 740 nm. pH may affect the visual characteristics an indicator because adding or taking away hydrogens from these compounds changes the conjugation of these compounds. Ultimately, changes in a molecule's chromophore are due to shifts in the energy of the molecule's highest occupied molecular orbital (HOMO) or lowest unoccupied molecular orbital (LUMO). For example, a pH indicator's HOMO and LUMO may be shifted upon protonation and deprotonation, resulting in the compound absorbing different wavelengths of light. The compound will then reflect or emit various wavelengths of light, based upon changes to the HOMO and LUMO.

pH indicators are often used in titrations, but they can also be useful in chemical synthesis, cell biology, medicine, and other fields. In aqueous solutions pH is based upon the negative log of hydronium ion concentration in a solution, or the negative log of hydrogen ion concentration under the Arrhenius model. pOH can also be probed. In aqueous solutions pOH is based upon the negative log of the hydroxide ion concentration. pH varies with temperature, but in general a neutral pH is 7. The body tightly regulates pH to ensure the occurrence of necessary chemical reactions. Physiological pH revolves around 7.365.

Often indicators change color over the span of one to two pH units. Generally we visually perceive the change in color of a solution, for example from yellow to pink, when about one in ten colorimetric compounds have a change in their chromophore. pH indicators vary in size, color, and variety, but the best pH indicators require a low concentration due to the brilliance of their chromophore. Helpful pH indicators are reversible over a number of pH changes and interfere minimally with the equilibrium of the reaction.

pH may be probed fairly precisely with the use of a pH meter. pH meters measure the pH of the solution electrically by measuring electrical potential as a function of ion concentration. Sometimes pH indicators are used in conjunction with a pH meter, or pH indicators can be used in conjunction with other pH indicators, such as in a pH strip.

### BRIEF SUMMARY OF THE INVENTION

It is an object of the invention to provide a low-cost indicator having advantages over previous indicators. It is also an object of the invention to provide an indicator that dissolves in a number of solvents and possesses a dynamic chromophore.

The indicator's dynamic chromophore may be due to solvatochromism. For instance, the indicator's chromophore may absorb and reflect electromagnetic radiation differently depending on the solvent or other molecules surrounding the indicator. In various embodiments the changes to the indicator's HOMO or LUMO may be due to electronic interactions of the calixarene cavity. In various embodiments the changes to the indicator's HOMO or LUMO may be due to supramolecular self-assembly.

A variety of methods may be used to incorporate a calixarene as an indicator. The calixarene may be used as a visual indicator. The calixarene may be used as a visual indicator for pH. The calixarene may be measured radiometrically. The calixarene may be measured photometrically. The calixarene may be a resorcinarene. The calixarene may be a pyrogallarene. The pyrogallarene may be a pyrogallol[4]arene. The pyrogallol[4]arene may be used to assess water content. The pyrogallol[4]arene may be used in its solid phase. The pyrogallol[4]arene may be used to assess metal content. The pyrogallol[4]arene may be used to assess pH. The pyrogallol[4]arene may change color in the range of pH 4 to 7. The pyrogallol[4]arene may be dissolved in a solution of acetone. The pyrogallol[4]arene may be saturated in a solution of methanol. A method of using a pyrogallol[4]arene as a pH indicator may also be accomplished in accordance with various embodiments. This embodiment may comprise dissolving the pyrogallol[4]arene in at least one solution and optically observing the color of the solution. A pyrogallarene solution may be prepared in accordance with various embodiments. The solution may comprise purified or recrystallized pyrogallol[4]arene and a solvent. In accordance with various embodiments the pyrogallarene solution may be larger than 100 mL. Further, the recrystallized pyrogallol[4]arene may be dissolved in the solvent of acetone or the solvent of methanol.

These and other objects and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates the chemical structure of a pyrogallol[4]arene.

FIG. 2 illustrates the chemical structure of a resorcin[4]arene.

FIG. 3 illustrates the chemical structure of a functionalized calixarene.

FIG. 4 illustrates the chemical structure of a calixarene.

FIG. 5 illustrates the chemical structure of a pyrogallarene.

FIG. 6 illustrates the chemical structure of a resorcinarene.

### DETAILED DESCRIPTION OF THE PREFERRED EMBODIMENTS

The description that follows describes, illustrates, and exemplifies one or more particular embodiments of the present invention in accordance with its principles. This description is not provided to limit the invention to the embodiment or embodiments described herein, but rather to explain and teach the principles of the invention in such a way to enable one of ordinary skill in the art to understand these principles and, with that understanding, be able to apply them to practice not only the embodiment or embodiments described herein, but also other embodiments that may come to mind in accordance with these principles.

The scope of the present invention is intended to cover all such embodiments that may fall within the scope of the appended claims, either literally or under the doctrine of equivalents.

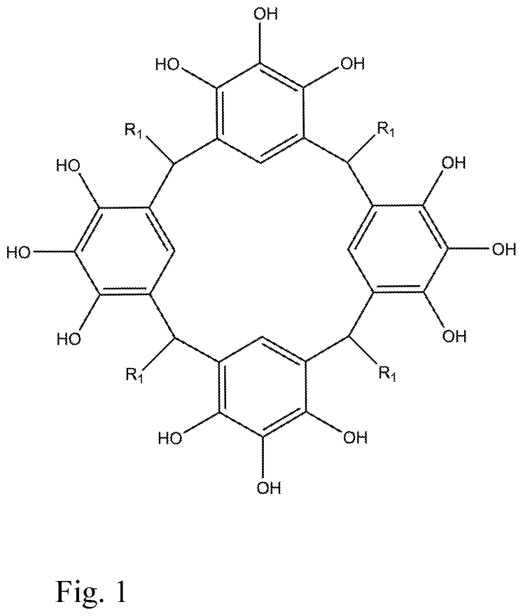

Referring to FIG. 1, a pyrogallol[4]arene is illustrated. Pyrogallol[4]arenes are commonly synthesized through an acid-catalyzed condensation reaction of 1:1 molar ratios of pyrogallol and an aldehyde. Each aldehyde adjoins two pyrogallols by way of the acid catalyzed condensation reaction. Upon completion of the condensation reaction the former aldehyde covalently links two pyrogallol molecules and is depicted as R₁ throughout the figures and in FIG. 1. R₁ is located in the meso position of the aromatic ring. Although R groups are commonly represented as organic chains, R₁ may be any pendant group, polar or nonpolar. R₁ may also be organic or inorganic. In at least one embodiment R₁ is a hydrocarbon chain. R₁ may be a propyl group (C₃H₇). R₁ may contain an alcohol. R₁ may contain a sulfoxide. R₁ may contain a sulfonate. R₁ may contain a carboxylic acid. R₁ may contain an amine. R₁ may contain a halogen. R₁ may contain a ketone. R₁ may contain a nitrile, or R₁ may contain an ester.

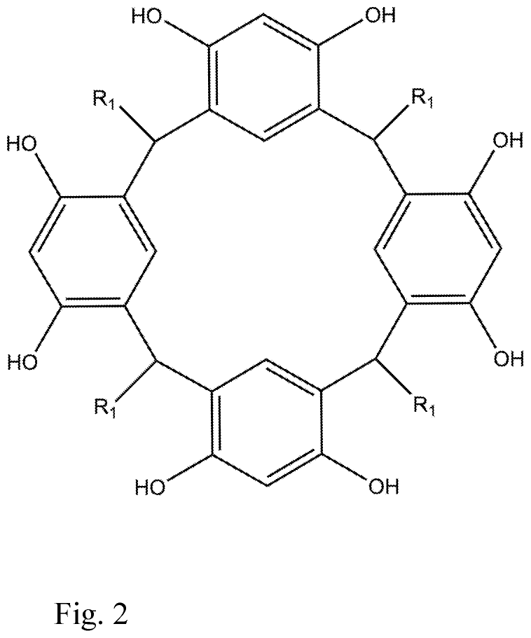

Referring to FIG. 2, a resorcin[4]arene is illustrated. Resorcin[4]arenes are commonly synthesized through an acid-catalyzed condensation reaction of 1:1 molar ratios of resorcinol and an aldehyde. Each aldehyde adjoins two resorcinols by way of the acid catalyzed condensation reaction. Upon completion of the condensation reaction the former aldehyde covalently links two resorcinol molecules and is depicted as R₁ in FIG. 2 and throughout the figures.

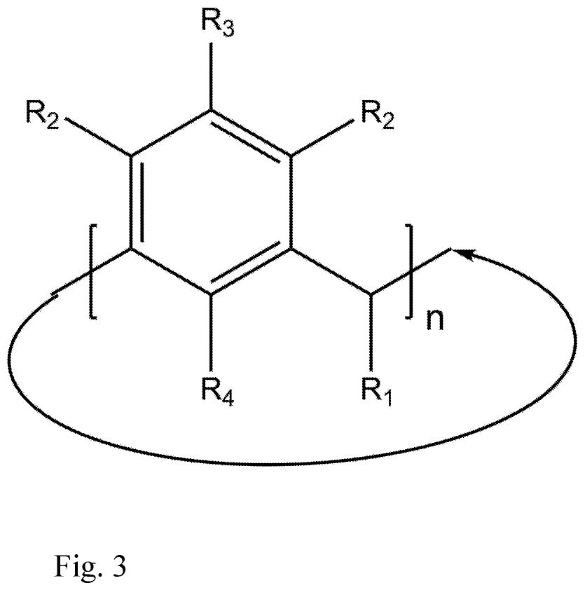

Referring to FIG. 3, a functionalized calixarene is illustrated. R₄ operates as a pendant group on the lower rim of the aromatic ring. Although R groups are commonly represented as organic chains, R₄ may be any pendant group, polar or nonpolar. R₄ may also be composed of organic or inorganic atoms. R₂ and R₃ are pendant groups on the upper rim of the aromatic ring. Like R₄ and R₁, R₂ and R₃ may be inorganic or organic, polar or nonpolar. In at least one embodiment R₂ and R₃ are OH or H groups. In at least one embodiment R₂ is a hydroxyl group and R₁ is a hydrogen. In at least one embodiment R₂ and R₃ are hydroxyl groups.

The ring size is designated by n. For example, where four aromatic rings are linked together the structure would be a calix[4]arene, and n would be equal to 4. If R₂ and R₃ are hydroxyl groups and R₄ is a hydrogen, making the aromatic ring pyrogallol, four pyrogallols linked together would be a pyrogallol[4]arene, and n would be equal to 4. By implication, if six pyrogallols are linked together the structure would be a pyrogallol[6]arene, and n would be equal to 6.

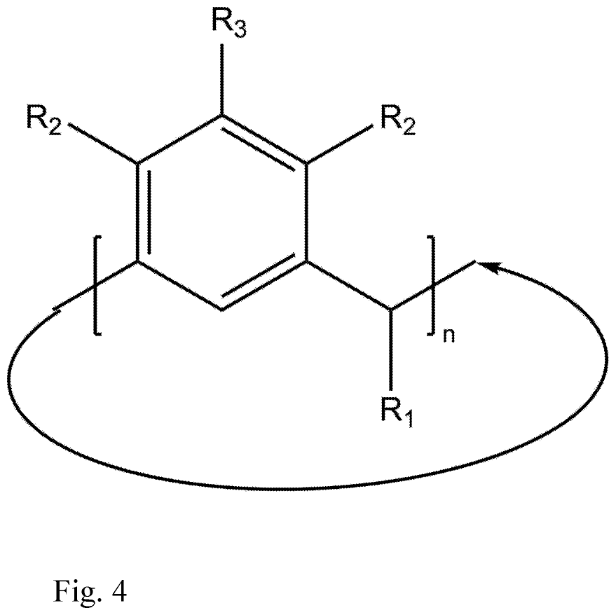

Referring to FIG. 4, a calixarene is illustrated. This calixarene differs from the calixarene in FIG. 3 because the lower rim of the aromatic ring lacks an R₄ group.

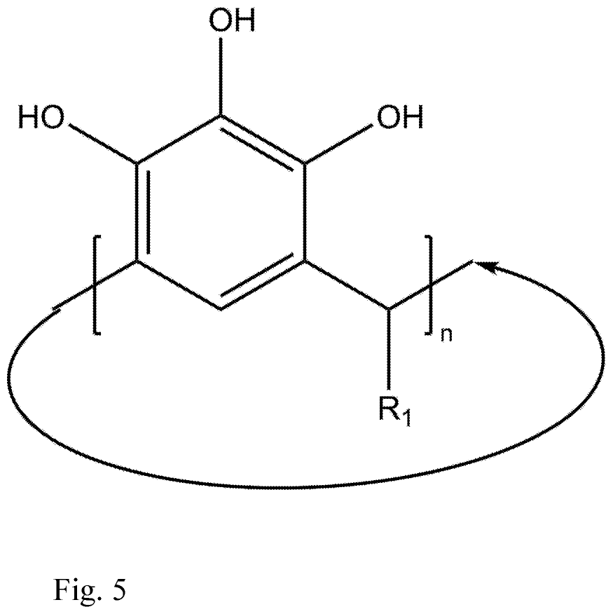

Referring to FIG. 5, a pyrogallarene is illustrated. Pyrogallols are linked together in a ring, n designating the number of pyrogallols linked. As in FIG. 1, a pendant group, R₁ operates to link the pyrogallol units. Where R₁ is a propyl group and n equals four the molecule would be C-propyl pyrogallol[4]arene.

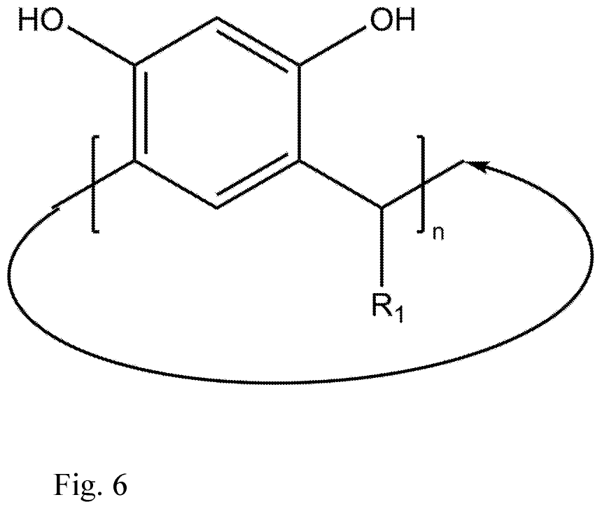

Referring to FIG. 6, a calixarene is illustrated. Resorcinols are linked together in a ring, n designating the number of resorcinols linked. As in FIG. 1, a pendant group, R₁ operates to link the resorcinol units.

In at least one embodiment of the invention a visual color occurs as a calixarene is dissolved in at least one solution. Where the pH is above or below the range of pH 3.5 to 6.5 the calixarene exhibits a differing color. In accordance with various embodiments a visible color change is observed as a calixarene is dissolved in at least one solution and the pH is adjusted in the range of pH 3.5 to 6.5.

In at least one embodiment the indication may be associated with the electronic interactions of the calixarene cavity. In at least one embodiment a visible color may be due to the supramolecular self-assembly of solvated pyrogallarene units. In accordance with various embodiments the solvated pyrogallarene unit may consist of three acetone molecules, three water molecules, and a pyrogallarene. In accordance with various embodiments the indicator may be dissolved in DMF, DMSO, Acetone, Methanol, Ethanol, or water. A solution containing the calixarene may be saturated with the calixarene. The calixarene could be dissolved in a nonpolar solvent, or the calixarene may be dissolved in a polar solvent. In at least one embodiment the calixarene is dissolved in a mixture of solvents. The calixarene can be incorporated in an indication strip, such as a pH strip.

The indicator's dynamic chromophore can be manifest in various ways. In at least one embodiment the indicator has at least one lambda max of 509 nm. In at least one embodiment the indicator has at least one lambda max in the range of 490 to 525 nm. In at least one embodiment the indicator has at least one lambda max in the range of 380 to 740 nm. In at least one embodiment the indicator has a lambda max in the range of 400 to 450 in a pH of 3. In at least one embodiment the indicator has a lambda max in the range of 640 to 700 nm in a neutral pH. In accordance with various embodiments the indicator has a variable lambda max. In at least one embodiment the indicator has a lambda max in the range of 600 to 640 nm. In at least one embodiment the indicator has a lambda max in the range of 400 to 450 nm. In at least one embodiment the indicator has a lambda max in the range of 450 to 480 nm.

Recrystallization of a pyrogallarene may be accomplished by dissolving the pyrogallarene in a solution and facilitating the recrystallization. The recrystallization solution may be methanol, ethanol, acetone, dmso, dmf, etc. The solution may be polar or nonpolar. Facilitating recrystallization may be accomplished by supersaturating the solution, sonicating the solution, providing nucleation sites for recrystallization, filtering a saturated solution, evaporating the solution, or any number of means. The resulting crystals may be vacuum dried, or the solvent may be separated from the crystals by other means, such as evaporation. The resulting crystals may then be dissolved in a solvent.

It will be appreciated by the person having skill in the art that the indicator disclosed herein may be used in a variety of circumstances. For example, the indicator may be used heterogeneously or homogeneously in at least one solution. Additionally, the indicator may be used to track supramolecular self-assembly or quantify aspects of self-assembly, for example relating to aqueous self-assembly. The person having ordinary skill in the art may appreciate the novelty of a calixarene indicator with a dynamic chromophore. For example, a pH indicator having a dynamic chromophore may be advantageous in comparison to various traditional pH indicators. Furthermore, the person having skill in the art may appreciate the ease of synthesis of the indicator compared to traditional indicators.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method of: using the chromophore of a calixarene as an indicator.

2. The method of claim 1, wherein the calixarene is used as a visual indicator.

3. The method of claim 2, wherein the calixarene is used as a visual indicator for pH.

4. The method of claim 1, wherein the calixarene is measured radiometrically.

5. The method of claim 1, wherein the calixarene is measured photometrically.

6. The method of claim 2, wherein the calixarene is a resorcinarene.

7. The method of claim 2, wherein the calixarene is a pyrogallarene.

8. The method of claim 7, wherein the pyrogallarene is a pyrogallol[4]arene.

9. The method of claim 8, wherein the pyrogallol[4]arene is used to assess water content.

10. The method of claim 8, wherein the pyrogallol[4]arene is used in a solid phase.

11. The method of claim 8, wherein the pyrogallol[4]arene is used to assess metal content.

12. The method of claim 8, wherein the pyrogallol[4]arene is used to assess pH.

13. The method of claim 12, wherein the pyrogallol[4]arene changes color in the range of about pH 4 to 7.

14. The method of claim 12, wherein the pyrogallol[4]arene is dissolved in at least one solution.

15. The method of claim 12, wherein the at least one solution is one of DMF, DMSO, Acetone, Methanol, Ethanol, or water.

16. A method of using the chromophore of a pyrogallol[4]arene as an indicator comprising:
dissolving the pyrogallol[4]arene in at least one solution; and
optically observing the color of the at least one solution.

17-20. (canceled)
